(19) United States
(12) Patent Application Publication
(10) Pub. No.: US 2019/0175510 A1
(43) Pub. Date: Jun. 13, 2019

(54) PHARMACEUTICAL MATRIX FORMULATIONS COMPRISING DIMETHYL FUMARATE

(71) Applicant: BIOGEN MA INC., Cambridge, MA (US)

(72) Inventors: Shyam B. Karki, Hillsborough, NJ (US); Peter Zawaneh, Brookline, MA (US); Cheuk-Yui Leung, Acton, MA (US); Yiqing Lin, Lexington, MA (US)

(21) Appl. No.: 16/275,739

(22) Filed: Feb. 14, 2019

Related U.S. Application Data
(63) Continuation of application No. 15/527,539, filed on May 17, 2017.
(60) Provisional application No. 62/081,907, filed on Nov. 19, 2014.

(51) Int. Cl. A61K 9/20; A61K 9/28; A61K 31/225; A61K 9/48; A61K 45/06

(52) U.S. Cl. CPC A61K 9/2054; A61K 9/282; A61K 9/2009; A61K 9/2013; A61K 9/2018; A61K 9/4808; A61K 9/2846; A61K 45/06; A61K 31/225

(57) ABSTRACT

The present invention provides novel pharmaceutical compositions of dimethyl fumarate. The pharmaceutical compositions of the present invention are in the form of a tablet and comprise one or more extended release polymer matrix. Also provided are pharmaceutical compositions in the form of a capsule comprising one or more tablets of the present invention. Methods of using the pharmaceutical compositions of the present invention for treating multiple sclerosis are also included.

### REFERENCE TO RELATED APPLICATIONS

This application is a continuation of U.S. application Ser. No. 15/527,539, filed on May 17, 2017, which is a U.S. national stage filing under 35 U.S.C. § 371 based on International Application PCT/US2015/061448, filed on Nov. 19, 2015, which claims the benefit of the filing date, under 35 U.S.C. § 119(e), of U.S. Provisional Application No. 62/081,907, filed on Nov. 19, 2014. The entire content of each of the foregoing applications, including all drawings, formulae, specification and claims, is incorporated herein by reference.

### BACKGROUND OF THE INVENTION

Tecfidera® (dimethyl fumarate) was approved by FDA in March, 2013 to be used for treating adults with relapsing forms of multiple sclerosis (MS). The starting dose for the currently approved formulation of Tecfidera® is 120 mg twice a day orally. After 7 days, the dose is increased to the maintenance dose of 240 mg twice a day orally.

Dimethyl fumarate (DMF) quickly gets absorbed in vivo and converted to monomethyl fumarate (MMF). The half-life of MMF was shown to be approximately 1 hour (0.9 h in rat at 100 mg/Kg oral dose). Both DMF and MMF are metabolized by esterases which are ubiquitous in the GI tract, blood and tissues.

DMF has demonstrated an acceptable safety profile in phase 3 clinical trials. However, tolerability issues such as flushing and gastrointestinal events were observed. While these events are generally mild to moderate in severity, it is desirable to reduce these side effects. It is also desirable to develop a once a day dosing formulation as opposed to the current twice a day formulation to improve patient compliance and convenience.

As such, there is a need for new pharmaceutical formulations of dimethyl fumarate with improved pharmacokinetic profiles and/or dosing regimen.

### SUMMARY OF THE INVENTION

The present invention provides novel pharmaceutical polymer matrix compositions of dimethyl fumarate that have pharmacokinetic profiles suitable for a once daily dosing regimen. The pharmaceutical compositions of the present invention have AUC and/or Cₘₐₓ that are comparable with the currently approved twice-a-day formulation. In addition, the pharmaceutical compositions of the present invention have a desirable extended release profile that may reduce the GI side effects observed for the current formulation.

Specifically, it has been surprisingly discovered that polymer matrix formulations in the form of a mini-tablet (e.g., with an average of the length and the width of the tablet in between 2 mm and 8 mm) have a more desirable extended release profile when compared with monolithic tablets (e.g., with an average of the length and the width of the tablet being 8 mm or bigger) and microtablets (i.e., having an average of the length and the width of the tablet being 2 mm or less). The 2 mm microtablets did not stay intact for an extended release system; while the 10 mm monolithic tablets did not achieve the required release profile. Dose dumping may also be an issue with monolithic tablets. In contrast, the minitablets of the present invention stayed intact long enough to function as an efficient extended release without the dose dumping, a potential issue with the monolithic tablets. The extended release profile of the present pharmaceutical compositions makes them suitable to be used in a once daily dosing regimen with potential reduced GI side effects observed for the current twice-a-day formulation.

In one embodiment, the pharmaceutical composition of the present invention is in the form of a tablet and comprises (i) dimethyl fumarate as the active substance present in the amount of 30-90% by weight of the tablet; and (ii) one or more extended release polymer matrix present in the amount of 1-70% by weight of the tablet, wherein the active substance is distributed throughout the matrix.

In another embodiment, the pharmaceutical composition of the present invention is in the form of a capsule comprising one of more tablets described above.

In yet another embodiment, the present invention provides a method of treating a subject having multiple sclerosis. The method comprises administering to the subject an effective amount of a pharmaceutical composition of the present invention described herein.

The present invention also provides a pharmaceutical composition described herein for use in treating a subject having multiple sclerosis.

Use of a pharmaceutical composition described herein for the manufacture of a medicament in treating multiple sclerosis is also included in the present invention.

### BRIEF DESCRIPTION OF THE DRAWINGS

FIG. 1. shows in vitro dissolution profiles for formulations A, B and C of the present invention using dissolution test 1.

FIG. 2. shows in vitro dissolution profiles for formulations A, B and C of the present invention using dissolution test 2.

FIG. 3. shows in vitro dissolution profiles for formulations A, B and C of the present invention using dissolution test 3.

FIG. 4 shows in vivo pharmacokinetic profile of formulation A as compared to currently approved Tecfidera formulation.

FIG. 5 shows in vivo pharmacokinetic profiles of formulation B and C.

### DETAILED DESCRIPTION OF THE INVENTION

In one embodiment, the pharmaceutic compositions of the present invention are in the form of a tablet, which comprises dimethyl fumarate as the active substance and one or more extended release polymer matrix, wherein the active substance is distributed throughout the matrix.

As used herein, the term “tablet” refers to a solid pharmaceutical dosage. Tablets of the instant pharmaceutical formulations can be made in any shape and size. In certain embodiments, the tablet has a shape that makes it easy and convenient for a patient to swallow, such as a tablet with a rounded or a rod-like shape without any sharp edge.

In one embodiment, the average of the length and the width of the tablets of the present invention is 2-10 mm, 2-9 mm, 2-8 mm, 2-7 mm or 2-6 mm. In another embodiment, the average of the length and the width is 2.5-7.5 mm, 2.5-7.0 mm, 2.5-6.5 mm, 2.5-6.0 mm, 2.5-5.5 mm, 2.5-5.0 mm, 2.5-4.5 mm, 3.0-7.0 mm, 3.0-6.5 mm, 3.0-6.0 mm, 3.0-5.5 mm, 3.0-5.0 mm. In another embodiment, the average of the length and the width is 3.1-4.9 mm, 3.2-4.8 mm, 3.3-4.7 mm, 3.4-4.6 mm, 3.5-4.5 mm, 3.6-4.4 mm, 3.7-4.3 mm, 3.8-4.2 mm, or 3.9-4.1 mm. In another embodiment, the average of the length and the width is 3.5 mm, 3.6 mm, 3.7 mm, 3.8 mm, 3.9 mm, 4.0 mm, 4.1 mm, 4.2 mm, 4.3 mm, 4.4 mm, or 4.5 mm. In yet another embodiment, the average of the length and the width is 4.0 mm. Alternatively, the average of the length and the width is 4.5-5.5 mm (e.g., 4.6 mm, 4.7 mm, 4.8 mm, 4.9 mm, 5.0 mm, 5.1 mm, 5.2 mm, 5.3 mm, 5.4 mm, or 5.5 mm), 4.6-5.4 mm, 4.7-5.3 mm, 4.8-5.2 mm, or 4.9-5.1 mm. In one embodiment, the average of the length and the width is 5.0 mm. In another alternative, the average of the length and the width is 5.5-6.5 mm (e.g., 5.5 mm, 5.6 mm, 5.7 mm, 5.8 mm, 5.9 mm, 6.0 mm, 6.1 mm, 6.2 mm, 6.3 mm, 6.4 mm or 6.5 mm), 5.6-6.4 mm, 5.7-6.3 mm, 5.8-6.2 mm, or 5.9-6.1 mm. In one embodiment, the average of the length and the width is 6.0 mm.

As used herein, the “length” refers to the dimension of the longest axis of the tablet and the “width” refers to the dimension of axis perpendicular to the longest axis in the largest plane of the tablet.

In one embodiment, the tablets of the present invention have a disk shape. The diameter of the disk can be between 2 mm and 10 mm. In one embodiment, the disk has a diameter between 2 mm and 8 mm. In another embodiment, the disk has a diameter between 2 mm and 6 mm (e.g., 2 mm, 3 mm, 4 mm, 5 mm or 6 mm). Alternatively, the disk has a diameter of 2 mm, 4 mm or 6 mm. In one embodiment, the pharmaceutical compositions of the present invention are in the form of a mini-tablet having a diameter of 4 mm.

In another embodiment, the tablets of the present invention have the shape of rectangular cuboid with angular or rounded edges. In one embodiment, the tablets have a rod shape.

The tablets of the present invention can also vary in thickness. In one embodiment, the tablet has a thickness of 1-3 mm. Alternatively, the tablet has a thickness of 1-2.5 mm or 1-2 mm.

As used herein, the “thickness” refers to the dimension of the axis that is perpendicular to the largest plane of the tablet.

As used herein, the “extended release polymer matrix” refers to a polymer matrix that releases the active substance dimethyl fumarate in a prolonged manner compared to the immediate-release formulations.

The term “prolonged” means that the active substance is released during a longer period of time than the current commercially available formulation of Tecfidera® (dimethyl fumarate), such as at least during a time period that is at least 1.2 times, at least 1.5 times, at least 2 times, at least 3 times, at least 4 times or at least 5 times greater than that of current commercial available formulation of Tecfidera®.

The extended release polymers that can be used in the pharmaceutical compositions described herein include, but are not limited to, hydroxylpropyl methyl cellulose (HPMC), ethyl cellulose (EC), hydroxypropyl cellulose (HPC), polyvinylpyrrolidone (PVP), polyethylene oxide (PEO), glyceryl monostearate, SoluPlus, polyvinyl alcohol (PVA), hydroxypropylmethylcellulose acetate succinate (HPMCAS), ethylene vinyl acetate (EVA), methacrylates (Eudragit™), cellulose acetate butyrate (CAB), cellulose acetate phthalate (CAP), poly(ethylene glycol), poly(vinyl acetate) (PVAc), polylactide (PLA), polyglycolide (PGA), copolymers of PLA/PGA and polycaprolactone (PCL), polyvinylpyrrolidone-co-vinyl acetate (Kollidon VA-64), polyrethanes, poly(lactic acid), poly(glycolic acid), poly(anhydride-imides), Poly(anhydride-esters), poly(iminocarbonates), poly(phosphazenes), poly(phosphoesters), alginic acid, carbomer copolymer, carbomer homopolymer, carbomer interpolymer, carboxymethylcellulose sodium, carrageenan, cellaburate, ethylcellulose aqueous dispersion, ethylcellulose dispersion type B, glyceryl monooleate, guar gum, hydroxypropyl betadex, polyvinyl acetate dispersion, shellac, sodium alginate, starch, pregelatinized starch and pregelatinized modified xanthan gum.

In one embodiment, the extended release polymer is hydroxylpropyl methyl cellulose (HPMC).

In certain embodiments, for the pharmaceutical tablet compositions described herein, 30-90% by weight of the tablet is dimethyl fumarate. More specifically, 40-80% by weight of the tablet is dimethyl fumarate. Even more specifically, 60-70% (e.g., 60%, 61%, 62%, 63%, 64%, 65%, 66%, 67%, 68%, 69% or 70%) by weight of the tablet is dimethyl fumarate. In a even more specific embodiment, the active substance dimethyl fumarate is present in the amount of 65% by weight of the tablet.

In certain embodiments, for the pharmaceutical tablet compositions described herein, the extended release polymer is present in the amount of 1-70% by weight of the tablet. More specifically, the extended release polymer is present in the amount of 1-25% or 5-20% by weight of the tablet. Even more specifically, the extended release polymer is present in the amount of 10-20% (e.g., 10%, 11%, 12%, 13%, 14%, 15%, 16%, 17%, 18%, 19% or 20%) by weight of the tablet. In a even more specific embodiment, the extended release polymer is present in the amount of 10% by weight of the tablet. Alternatively, the extended release polymer is present in the amount of 13% by weight of the tablet. In another alternative, the extended release polymer is present in the amount of 17% by weight of the tablet.

As used herein, the term “% by weight of the tablet” refers to the percentage by weight of each ingredient in the core tablet, excluding any exterior coatings.

In one embodiment, the pharmaceutical tablet composition described herein comprises dimethyl fumarate in the amount of 40-80% by weight of the tablet and an extended release polymer described herein in the amount of 1-25% by weight of the tablet.

In yet another embodiment, the pharmaceutical tablet composition described herein comprises dimethyl fumarate in the amount of 60-70% by weight of the tablet and an extended release polymer described herein in the amount of 10-20% by weight of the tablet.

The tablets of the present invention can include other pharmaceutically acceptable excipients, such as fillers, lubricants, glidants and etc.

In certain embodiments, the tablets described herein also comprise one or more fillers. Exemplary fillers that can be used in the present invention include, but are not limited to, hydroxypropyl methylcellulose (HPMC), hydroxypropyl cellulose (HPC), polyvinylpyrrolidone (PVP), polyethylene oxide, methyl cellulose, ethyl cellulose, sodium carboxy methyl cellulose, polyethylene glycol (PEG), polyvinyl alcohols, polymethacrylates, starch paste, sodium starch, acacia, tragacanth, gelatin, alginate, sodium alginate, alginic acid, cellulose, candelilla wax, carnuba wax, copolyvidone, glyceryl behenate, lactose hydrous, microcrystalline cellulose (MCC), mannitol, calcium phosphate, sucrose, sorbitol, xylitol, amino methacrylate copolymer, ammonio methacrylate copolymer, ammonio methacrylate copolymer dispersion, calcium carbonate, calcium phosphate dibasic anhydrous, calcium phosphate dibasic dehydrate, calcium phosphate tribasic, calcium sulfate, cellaburate, silicified microcrystalline cellulose, powdered cellulose, cellulose acetate, corn syrup, corn syrup solids, dextrates, dextrin, dextrose, dextrose excipient, erythritol, ethyl acrylate and methyl methacrylate copolymer dispersion, fructose, isomalt, kaolin, alpha-lactalbumin, lactitol, lactose anhydrous, lactose monohydrate, magnesium carbonate, magnesium oxide, maltitol, maltodextrin, maltose, methacrylic acid copolymer, methacrylic acid copolymer dispersion, methacrylic acid and ethyl acrylate copolymer dispersion, polydextrose, polyethylene glycol, propylene glycol monocaprylate, pullulan, simethicone, sodium chloride, pregelatinized starch, pregelatinized modified starch, corn starch, hydroxypropyl corn starch, pregelatinized hydroxypropyl corn starch, pea starch, hydroxypropyl pea starch, pregelatinized hydroxypropyl pea starch, potato starch, hydroxypropyl potato starch, pregelatinized hydroxypropyl potato starch, tapioca starch, wheat starch, hydrogenated starch hydrolysate, compressible sugar, Confectioner's sugar, Talc and trehalose. In a specific embodiment, the filler is lactose.

The filler can be present in the amount of 1-50%, 10-40% or 20-30% by weight of the tablet. More specifically, the filler can be present in the amount of 20-25% (e.g., 20%, 21%, 22%, 23%, 24% or 25%) by weight of the tablet.

The tablets of the present invention described herein can also include one or more lubricants. Exemplary lubricants include, but are not limited to, behenoyl polyoxylglycerides, calcium stearate, hydrogenated castor oil, hydrogenated coconut oil, glyceryl behenate, glyceryl monostearate, glyceryl tristearate, lauric acid NF32, magnesium stearate, light mineral oil, myristic acid, hydrogenated palm oil, palmitic acid, poloxamer, polyethylene glycol, polyoxyl 10 oleyl ether, polyoxyl 15 hydroxystearate, polyoxyl 20 cetostearyl ether, polyoxyl 35 castor oil, hydrogenated polyoxyl 40 castor oil, polyoxyl 40 stearate, polysorbate 20, polysorbate 40, polysorbate 60, polysorbate 80, potassium benzoate, sodium benzoate, sodium lauryl sulfate, sodium stearate, sodium stearyl fumarate, sorbitan monolaurate, sorbitan monooleate, sorbitan monopalmitate, sorbitan monostearate, sorbitan sesquioleate, sorbitan trioleate, stearic acid, stearic acid, purified sucrose stearate, Talc, hydrogenated vegetable oil type I and zinc stearate. In a specific embodiment, the lubricant is magnesium stearate.

In certain embodiments, the lubricant is present in the amount of 0.1-10%, 0.1-5% or 0.1-1% (e.g., 0.1%, 0.2%, 0.3%, 0.4%, 0.6%, 0.7%, 0.8%, 0.9%, or 1.0%) by weight of the tablet. In a specific embodiment, the lubricant is present in the amount of 0.5% by weight of the tablet.

The tablets of the present invention can also include one or more glidants. Exemplary glidants include, but are not limited to, calcium phosphate tribasic, calcium silicate, powdered cellulose, magnesium oxide, magnesium silicate, magnesium trisilicate, dental-type silica, silicon dioxide, hydrophobic colloidal silica, colloidal silicon dioxide, fumed silicon dioxide, sodium stearate and Talc. In a specific embodiment, the glidant is silicon dioxide (e.g., Aerosil®).

In certain embodiments, the glidant is present in the amount of 0.1-10%, 0.1-5% or 0.1-1% (e.g., 0.1%, 0.2%, 0.3%, 0.4%, 0.6%, 0.7%, 0.8%, 0.9%, or 1.0%) by weight of the tablet. In a specific embodiment, the lubricant is present in the amount of 0.5% by weight of the tablet.

The tablets of the present invention can be further coated with an enteric coating. As used herein, “enteric coating” refers to a coating that is stable at the highly acidic pH (e.g., pH˜3) found in the stomach, but breaks down rapidly at a less acidic pH (e.g., pH 7-9). Any enteric coating material known in the art can be used in the present invention.

In certain embodiment, the enteric coating comprises an excipient selected from the group consisting of a copolymer of methacrylic acid and methyl methacrylate, a copolymer of methacrylic acid and ethyl acrylate, hypromellose phthalate (HPMCP), cellulose acetate phthalate. More specifically, the enteric coating comprises a copolymer of methacrylic acid and methyl methacrylate. Even more specifically, the ratio of methacrylic acid to methyl methacrylate in the copolymer is 0.8:1 to 1.2:1, (e.g., 1:1). In an even more specific embodiment, the enteric coating comprises EUDRAGIT® L 100 (poly(methacylic acid-co-methyl methacrylate) 1:1).

In certain embodiments, the enteric coating of the present invention further comprises one or more plasticizers. Exemplary plasticizer include, but are not limited to, acetyltriethyl citrate, benzyl benzoate, castor oil, chlorobutanol, diacetylated monoglycerides, dibutyl sebacate, diethyl phthalate, glycerin, mannitol, polyethylene glycol, polyethylene glycol monomethyl ether, propylene glycol, pullulan, sorbitol, sorbitol sorbitan solution, triacetin, tributyl citrate, triethyl citrate and Vitamin E. In a more specific embodiment, the plasticizer is triethyl citrate.

In one embodiment, the enteric coating of the present invention comprises EUDRAGIT® L 100 and triethyl citrate. More specifically, the molar ratio of the triethyl citrate to EUDRAGIT® L 100 is from 1:1 to 1:20. Even more specifically, the molar ratio of the triethyl citrate to EUDRAGIT® L 100 is 1:5.

In certain embodiments, for the tablets of the present invention, the enteric coating is present in the amount of 1-20% or 5-15% by weight of the tablet. The weight of the tablet is the total weight of the core tablet, excluding any exterior coating, such as the enteric coating. More specifically, the enteric coating is present in the amount of 10-15% (e.g., 10%, 11%, 12%, 13% or 15%) by weight of the tablet. Even more specifically, the enteric coating is present in the amount of 12% by weight of the tablet.

In one embodiment, the pharmaceutical tablet composition of the present invention comprises dimethyl fumarate in the amount of 40-80% by weight of the tablet and an extended release polymer described herein in the amount of 1-25% by weight of the tablet, wherein the tablet is further coated with an enteric coating in the amount of 1-20% by weight of the tablet. More specifically, the extended release polymer is HPMC and the enteric coating comprises EUDRAGIT® L 100 and triethyl citrate. Even more specifically, the molar ratio of EUDRAGIT® L 100 to triethyl citrate is 5:1. In an even more specific embodiment, the tablet has a diameter of 4-8 mm, preferably 4-6 mm, more preferably 4 mm.

In yet another embodiment, the pharmaceutical tablet composition described herein comprises dimethyl fumarate in the amount of 60-70% by weight of the tablet and an extended release polymer described herein in the amount of 10-15% by weight of the tablet, wherein the tablet is further coated with an enteric coating in the amount of 10-15% by weight of the tablet. More specifically, the extended release polymer is HPMC and the enteric coating comprises EUDRAGIT® L 100 and triethyl citrate. Even more specifically, the molar ratio of EUDRAGIT® L 100 to triethyl citrate is 5:1. In an even more specific embodiment, the tablet has a diameter of 4-8 mm, preferably 4-6 mm, more preferably 4 mm.

In one embodiment, the pharmaceutical composition of the present invention is in the form of a tablet having a diameter of 4 mm, which comprises (i) dimethyl fumarate as an active substance, wherein the active substance is present in the amount of 64%-66% by weight of the tablet, (ii) a filler in the amount of 23-25% by weight of the tablet; and (iii) one or more extended release polymer matrix present in the amount of 9%-11% by weight of the tablet, wherein the extended release polymer is HPMC and the active substance is distributed throughout the matrix, and wherein the tablet is coated with an enteric coating comprising a copolymer of methacrylic acid and methyl methacrylate, wherein the ratio of methacrylic acid to methyl methacrylate is 1:1 and the weight percentage of the enteric coating is 11-13% of the weight of the tablet. More specifically, the enteric coating comprises triethyl citrate as plasticizer and the molar ratio of triethyl citrate to the copolymer of methacrylic acid and methyl methacrylate is 1:5. Even more specifically, the filler is lactose (e.g., Flowlac). The pharmaceutical composition can further comprises one or more lubricants described herein and one or more glidants described herein. More specifically, the lubricant is magnesium stearate and the glidant is silicon dioxide (e.g., Aerosil).

In another embodiment, the pharmaceutical composition of the present invention is in the form of a tablet having a diameter of 4 mm, which comprises (i) dimethyl fumarate as an active substance, wherein the active substance is present in the amount of 64%-66% by weight of the tablet, (ii) a filler in the amount of 20-22% by weight of the tablet; and (ii) one or more extended release polymer matrix present in the amount of 12%-14% by weight of the tablet, wherein the extended release polymer is HPMC and the active substance is distributed throughout the matrix, and wherein the tablet is coated with an enteric coating comprising a copolymer of methacrylic acid and methyl methacrylate, wherein the ratio of methacrylic acid to methyl methacrylate is 1:1 and the weight percentage of the enteric coating is 11-13% of the weight of the tablet. More specifically, the enteric coating comprises triethyl citrate as plasticizer and the molar ratio of triethyl citrate to the copolymer of methacrylic acid and methyl methacrylate is 1:5. Even more specifically, the filler is lactose (e.g., Flowlac). The pharmaceutical composition can further comprises one or more lubricants described herein and one or more glidants described herein. More specifically, the lubricant is magnesium stearate and the glidant is silicon dioxide (e.g., Aerosil).

The tablets of the present invention provide extended release of the active substance dimethyl fumarate when subjected to a dissolution test. The dissolution test can be carried out according to standard procedures published by USP-NF.

In one embodiment, the dissolution profile of the tablets of the present invention is determined by subjecting the tablets to an in vitro dissolution test employing 0.1 N hydrochloric acid as dissolution medium during the first 2 hours of the test and then USP Simulated Intestinal Fluid (SIF) without pancreatin as dissolution medium in a USP Apparatus II (paddle apparatus) (Test 1). Alternatively, the dissolution profile is determined by subjecting the tablets of the present invention to an in vitro dissolution test employing USP Simulated Gastric Fluid (SGF) without pepsin as dissolution medium during the first 2 hours of the test and then USP Simularted Intestinal Fluid (SIF) without pancreatin as dissolution medium in a USP Apparatus IV (flow-through cell) (Test 2). In yet another alternative, the dissolution profile is determined by subjecting the tablets of the present invention to an in vitro dissolution test employing USP Simularted Intestinal Fluid (SIF) without pancreatin in a USP Apparatus IV (flow-through cell) (Test 3). USP SIF and SGF solutions can be prepared according to according to procedures described in USP35-NF30.

In certain embodiments, when subjected to dissolution Test 1, the tablet composition of the present invention has the following dissolution profile:

within the first 2 hours of the test, less than 10% by weight of the active substance in the tablet is released;

within the first 4 hours of the test, 30-70% by weight of the active substance in the tablet is released; and

within the first 7 hours of the test, 50-100% by weight of the active substance in the tablet is released.

In certain embodiments, when subjected to dissolution Test 1, the tablet composition of the present invention has the following dissolution profile:

within the first 2 hours of the test, less than 10% by weight of the active substance in the tablet is released;

within the first 4 hours of the test, 50-70% by weight of the active substance in the tablet is released; and

within the first 7 hours of the test, 90-100% by weight of the active substance in the tablet is released.

In certain embodiments, when subjected to dissolution Test 2, the tablet composition of the present invention has the following dissolution profile:

within the first 2 hours of the test, less than 10% by weight of the active substance in the tablet is released;

within the first 4 hours of the test, 15-25% by weight of the active substance in the tablet is released; and

within the first 9 hours of the test, 50-100% by weight of the active substance in the table is released.

In certain embodiments, the pharmaceutical composition of the present invention releases 80% of dimethyl fumarate from the composition within 3-10 hours, preferably within 4-8 hours, more preferably within 4-6 hours in an in vivo pharmacokinetic study. In particular, dogs were administerd with the pharmaceutical composition of the present invention containing 240 mg of DMF.

The present invention also provides a pharmaceutical composition in the form of a capsule comprising one or more tablets described herein. In one embodiment, the capsule comprises 5-30 tablets. More specifically, the capsule comprises 14 to 20 tablets, such as 14, 15, 16, 17, 18, 19 or 20 tablets. Even more specifically, the capsule comprises 16 tablets.

In certain embodiments, the amount of dimethyl fumarate in the pharmaceutical composition described herein is from 10 mg to 960 mg, more specifically, from 15 mg to 480 mg. In certain embodiment, the amount of dimethyl fumarate in a single tablet described herein is from 10 mg to 50 mg. More specifically, the amount of dimethyl fumarate in a single tablet described herein is 15 mg. Alternatively, the amount of dimethyl fumarate in a single tablet described herein is 30 mg. In yet another embodiment, the amount of dimethyl fumarate in a single capsule described herein is from 90 mg to 960 mg, more specifically from 120 mg to 480 mg. In one embodiment, the amount of dimethyl fumarate in a single capsule described herein is 240 mg. Alternatively, the amount of dimethyl fumarate in a single capsule described herein is 480 mg.

The present invention also provides a method of treating a subject having multiple sclerosis (e.g., relapsing-remitting MS, secondary progressive MS, primary progressive MS, progressive relapsing MS) comprising administering to the subject an effective amount of a pharmaceutical composition described herein. In one embodiment, the method of the present invention is for treating relapsing-remitting MS.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

As used herein, the term “subject” and the term “patient” can be used interchangeable and they refer to a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The effective amount or therapeutic dosage of the pharmaceutical compositions described herein that is administered to treat a patient depends on a number of factors, which include, but are not limited to, weight and age of the patient, route of administration, the underlying causes of the disease to be treated, and the severity of the disease to be treated. In one embodiment, the effective dosage can range from 1 mg/kg to 50 mg/kg (e.g., from 2.5 mg/kg to 20 mg/kg or from 2.5 mg/kg to 15 mg/kg). In one embodiment, an effective amount of DMF to be administered to a subject, for example orally, can be from 0.1 g to 1 g per day, for example, from 200 mg to 800 mg per day (e.g., from 240 mg to 720 mg per day; or from 480 mg to 720 mg per day; or 480 mg per day; or 720 mg per day).

The daily dose can range, but is not limited to, a total amount of 60 mg to 800 mg, 60 mg to 720 mg, 60 mg to 500 mg, 60 mg to 480 mg, 60 mg to 420 mg, 60 mg to 360 mg, 60 mg to 240 mg, 60 mg to 220 mg, 60 mg to 200 mg, 60 mg to 180 mg, 60 mg to 160 mg, 60 mg to 140 mg, 60 mg to 120 mg, 60 mg to 100 mg, 60 mg to 80 mg, 80 mg to 480 mg, 100 mg to 480 mg, 120 mg to 480 mg, 140 mg to 480 mg, 160 mg to 480 mg, 180 mg to 480 mg, 200 mg to 480 mg, 220 mg to 480 mg, 240 mg to 480 mg, 300 mg to 480 mg, 360 mg to 480 mg, 400 mg to 480 mg, 450 mg to 500 mg, 480 mg to 500 mg, 80 to 400 mg, 100 to 300 mg, 120 to 180 mg, or 140 mg to 160 mg.

In one embodiment, the daily dosage is 240 mg. Alternatively, the daily dosage is 480 mg.

The daily dose(s) of DMF may be administered in a single administration or in separate administrations of 2, 3, 4, or 6 equal doses. In one embodiment, the effective daily dose is 480 mg per day and is administered in one dose to a subject in need thereof. In another embodiment, the effective daily dose is 240 mg per day and is administered in one dose to a subject in need thereof.

In one embodiment, the pharmaceutical composition of the present invention is administered at least one hour before or after food is consumed by the subject in need thereof. In case the subject experiences side effects (e.g., flushing or GI discomfort), the subject can consume food shortly (e.g., 30 mins to an hour) before administered the pharmaceutical composition.

In one embodiment, the subject administered the pharmaceutical compositions of the present invention may take one or more non-steroidal anti-inflammatory drugs (e.g., aspirin) before (for example, 10 minutes to an hour, e.g., 30 minutes before) taking the pharmaceutical composition. In one embodiment, the subject administered the pharmaceutical composition takes the one or more non-steroidal anti-inflammatory drugs (e.g., aspirin) to control side effects (e.g., flushing). In another embodiment, the one or more non-steroidal anti-inflammatory drugs is selected from a group consisting of aspirin, ibuprofen, naproxen, ketoprofen, celecoxib, MK-0524, and combinations thereof. The one or more non-steroidal anti-inflammatory drugs can be administered in an amount of 50 mg to 500 mg before taking the dosage form described above. In one embodiment, a subject takes 325 mg aspirin before taking each dosage form described above.

In one embodiment, the subject in need of the treatment is administered a first dose of the pharmaceutical compositions described herein for a first dosing period; and administered a second dose of the pharmaceutical compositions described herein for a second dosing period. In one embodiment, the first dose is lower than the second dose (e.g., the first dose is half of the second dose). In one embodiment, the first dosing period is at least one week (e.g., 1-4 weeks). In one embodiment, the first dose of the pharmaceutical compositions comprises 240 mg of DMF and the pharmaceutical composition is administered to the subject once daily for the first dosing period. In one embodiment, the second dose of the pharmaceutical composition comprises 480 mg of DMF and the pharmaceutical composition is administered to the subject once daily for the second dosing period. In one embodiment, if the subject, after being administered the dose at the second dosing period, experiences more than expected level of side effects (e.g., flushing or a gastrointestinal disturbance), the subject can use a lower dose (e.g., the dose at the first dosing period) for a period (e.g., 1-4 weeks or more) sufficient to allow the side effects to decrease before returning to the dose at the second dosing period.

In one embodiment, the first dose of the pharmaceutical composition comprises 240 mg of DMF and the pharmaceutical composition is administered to the subject once daily for at least one week, and the second dose of the pharmaceutical composition comprises 480 mg of DMF and the pharmaceutical composition is administered to the subject once daily for at least two weeks.

In one embodiment, the subject is administered a first dose for one week and a second dose for a second dosing period of at least 48 weeks. In another embodiment, the subject is administered a first dose for one week and a second dose for a second dosing period of at least two years. In another embodiment, the subject is administered a first dose for one week and a second dose until the subject does not require treatment.

In certain embodiments, the methods of treating a subject having multiple sclerosis described herein furthter comprises adminstering to the subject a second therapeutic agent.

In one embodiment, the second therapeutic agents is a disease modifying agent. In one embodiment, the second therapeutic agents alleviate the side effects of dimethyl fumarate. For example, the second therapeutic agent can be a therapeutic agent that can reduce the flushing (e.g., aspirin) or GI disturbance (e.g., loperamide).

In another embodiment, the second therapetic agent is a Nrf-2 modulator.

In yet another embodiment, the second therapeutic agents can be, e.g., interferon beta-la (Avonex®, Rebif®), glatiramer (Copaxone®), modafinil, azathioprine, predisolone, mycophenolate, mofetil, mitoxantrone, natalizumab (Tysabri®), sphinogosie-1 phosphate modulator e.g., fingolimod (Gilenya®), and other drugs useful for MS treatment such as teriflunornide (Aubagio®), piroxicam, and phenidone.

The pharmaceutical DMF compositions of the present invention and the second therapeutic agent may be administered concurrently (as separate compositions or together in a single dosage form) or consecutively over overlapping or non-overlapping intervals. In the sequential administration, the DMF composition and the second therapeutic agent can be administered in any order. In some embodiments, the length of an overlapping interval is more than 2, 4, 6, 12, 24, 48 weeks or longer.

In order that the invention described herein may be more fully understood, the following examples are set forth. It should be understood that these examples are for illustrative purposes only and are not to be construed as limiting this invention in any manner.

### EXAMPLES

### Example 1. Methods for Preparing Pharmaceutical Compositions of The Present Invention

The API dimethyl fumarate is first blended with filler, glidant, lubricant and the extended release polymer in a blender for a predetermined period of time, for example, for 15 minutes. The blended powder is then compressed using a tablet press. Finally, the tablets are enteric coated for acid protection using a fluid bed granulator with a Wurster coating insert.

The following pharmaceutical compositions were prepared using the method described above. Formulation A is a microtablet formulation having a diameter of 2 mm and thickness of about 2.3 mm. Formulations B and C are mini-tablet formulations having 4 mm in diameter and about 1.8 mm in thickness. Percentage indicated in the tables are weight percentages. All three formulations are coated with enteric coating in the amount of 12% by weight of the tablet. The enteric coating comprises Eudragit L100 and triethyl citrate in a molar ratio of 5:1.

TABLE 1

| Formulations | Formulation A | Formulation B | Formulation C |
|---|---|---|---|
| DMF | 65% | 65% | 65% |
| Flowlac | 17% | 24% | 21% |
| HPMC K15M | 17% | 10% | 13% |
| MgSt | 0.5% | 0.5% | 0.5% |
| Aerosil | 0.5% | 0.5% | 0.5% |

### Example 2. In Vitro Dissolution Profiles

The in vitro dissolution profiles of the present pharmaceutical composition were determined according to methods described below, which are standard procedures published by USP-NF using USP apparatus II and IV.

Test 1. The pharmaceutical compositions of the present invention were subjected to an in vitro dissolution test employing 0.1 N hydrochloric acid as dissolution medium during the first 2 hours of the test and then USP Simulated Intestinal Fluid (SIF) without pancreatin as dissolution medium in a USP Apparatus II (paddle apparatus).

Test 2. The pharmaceutical compositions of the present invention were subjected to an in vitro dissolution test employing USP Simulated Gastric Fluid (SGF) without pepsin as dissolution medium during the first 2 hours of the test and then USP Simularted Intestinal Fluid (SIF) without pancreatin as dissolution medium in a USP Apparatus IV (flow-through cell).

Test 3. The pharmaceutical compositions of the present invention were subjected to an in vitro dissolution test employing USP Simularted Intestinal Fluid (SIF) without pancreatin as dissolution medium in a USP Apparatus IV (flow-through cell).

USP SIF solution can be prepared according to according to procedures described in USP35-NF30. For 1 L scale, the SIF solution can be prepared by dissolving 6.8 g of monobasic potassium phosphate in 250 mL of water followed by mixing. 77 mL of 0.2 N sodium hydroxide and 500 mL of water are added sequentially. The pH of the resulting solution is adjusted with either 0.2 N sodium hydroxide or 0.2 N hydrochloric acid to a pH of 6.8±0.1 followed by dilution with water to 1000 mL. USP SGF solution can be prepared according to procedures described in USP35-NF30. For 1 L scale, the SGF solution can be prepared by dissolving 2.0 g of sodium chloride (NaCl) in 7.0 mL of hydrochloric acid (HCl) and sufficient water to make 1000 mL.

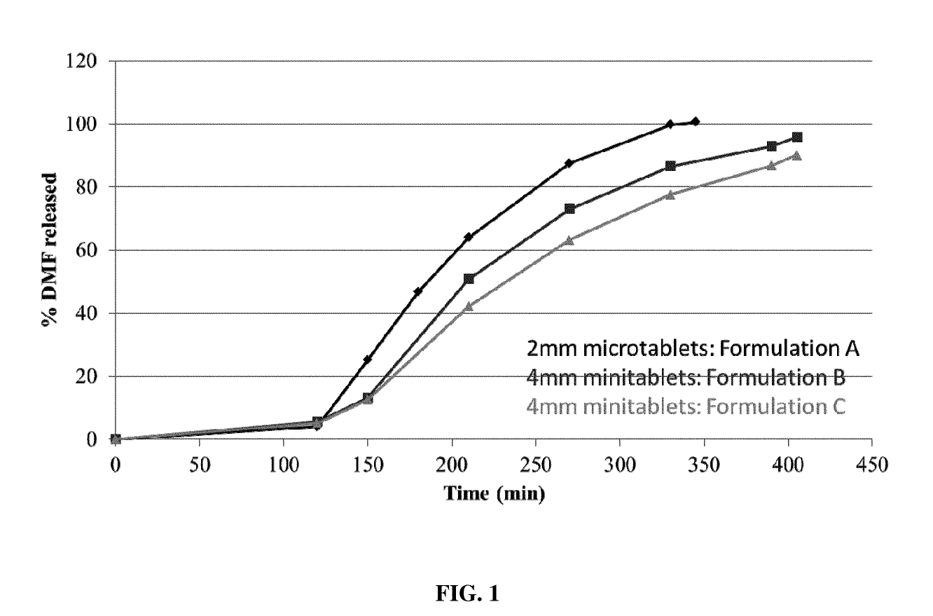

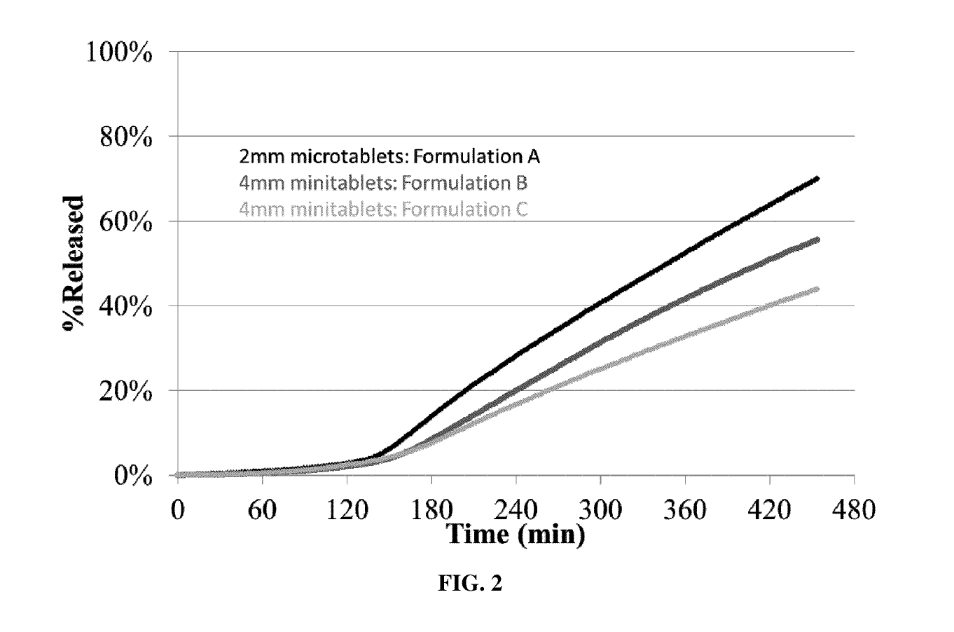

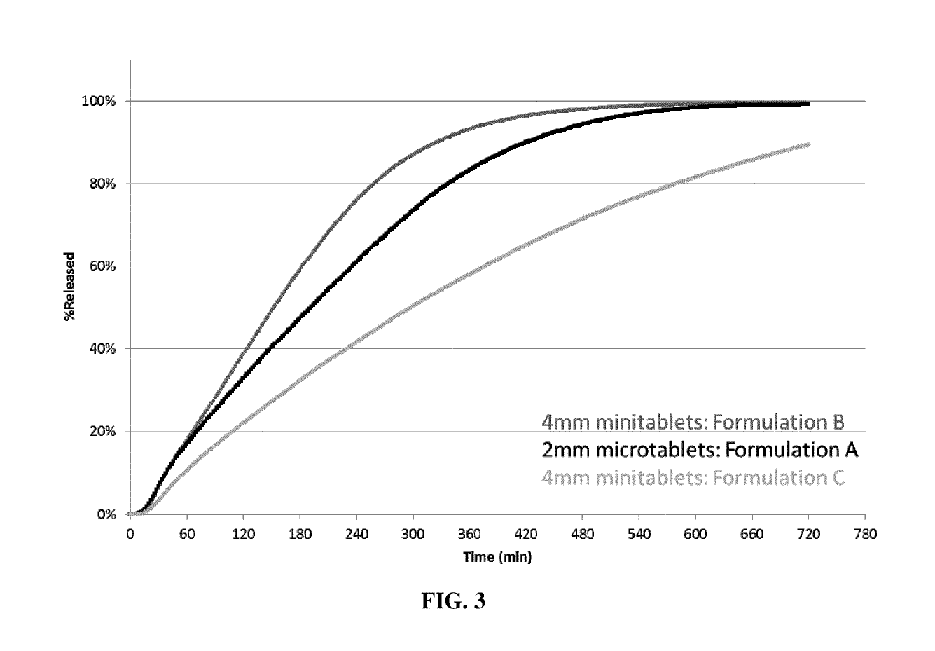

The dissolution profiles for Formulations A, B and C determined are shown in FIG. 1 (using Test 1), FIG. 2 (using Test 2) and FIG. 3 (using Test 3). All three formulations show extended release in vitro dissolution profiles. The 2 mm microtablet Formulation A has a faster release profile than the 4 mm mini-tablet Formulations B and C.

### Example 3. In Vivo Pharmacokinetic Profiles

Formulations A, B and C were selected for a dog PK study.

Male dogs were divided into six test groups and 1 control group with 4 dogs in each group. Dogs in the control group were administered with currently approved Tecfidera® formulation. Dogs in the test groups were administered with Formulations D, E or F or other DMF formulations. Dogs were fasted overnight until 1 hour post dose. 240 mg DMF in size 0 capsules were administered to the dogs orally, followed by approximately 10 mL of water. A second flush with approximately 10 mL of water may be administered if necessary to ensure capsule delivery.

Approximately 1 mL of blood was collected from each animal at 10 blood collection time points: predose and at 0.25, 0.5, 1, 2, 4, 8, 12, 16, and 24 hours postdose. Blood was collected via a jugular vein into tubes containing sodium heparin anticoagulant. Prior to blood collection, 40 μL of 250 mg/mL solution of aqueous sodium fluoride was added to each collection tube. The NaF solution may be prepared on the day prior to the study and stored refrigerated between uses and equilibrated to ambient temperature and vortexed prior to each use. The cephalic vein may be used as an alternative blood collection site.

At each protocol specified time point, 1 mL of blood was collected into a chilled sodium heparin/sodium fluoride tube and mixed immediately by gently inverting the tube

5 to 7 times to ensure uniform mixing. Avoid vigorous shaking to prevent hemolysis of
the blood sample. Place the blood sample into wet ice or cryorack. Centrifuge samples within 30 minutes of collection at 4° C. for 15 minutes at 1500×g. Plasma was aliquoted equally into 1.8 or 2 mL cryovials and be maintained on dry ice prior to storage at approximately −70° C.

Plasma was then analyzed. MMF in the plasma was quantified by LC-MS/MS with calibration range of 10 ng/ml-5000 ng/ml using ¹³C-MMF as internal standard. Plasma can be diluted with 1:10 dilution if necessary.

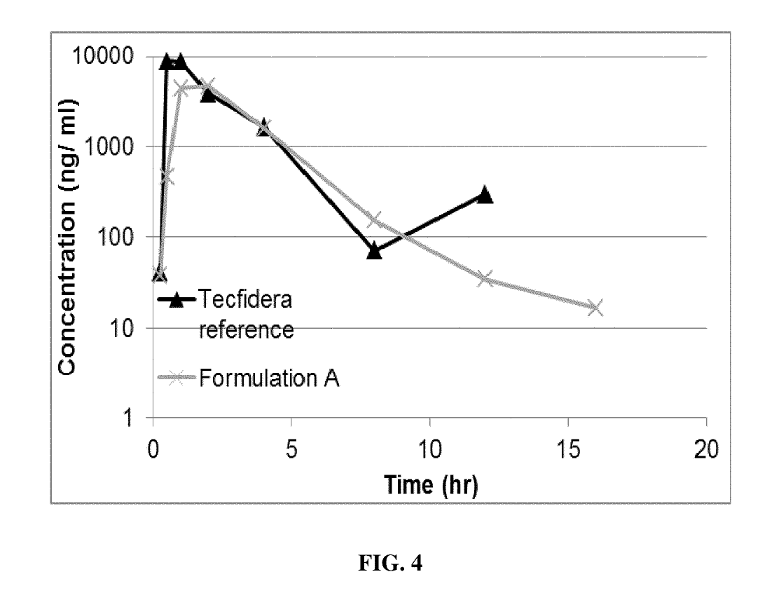

As shown in FIG. 4, the 2 mm microtablet Formulation A has a PK profile similar to the currently approved Tecfidera formulation, which is an immediate release tablet formulation with enteric coating. The data suggests that the 2 mm microtablets did not stay intact for an extended release system even the dissolution test shows a release profile of 6 hrs.

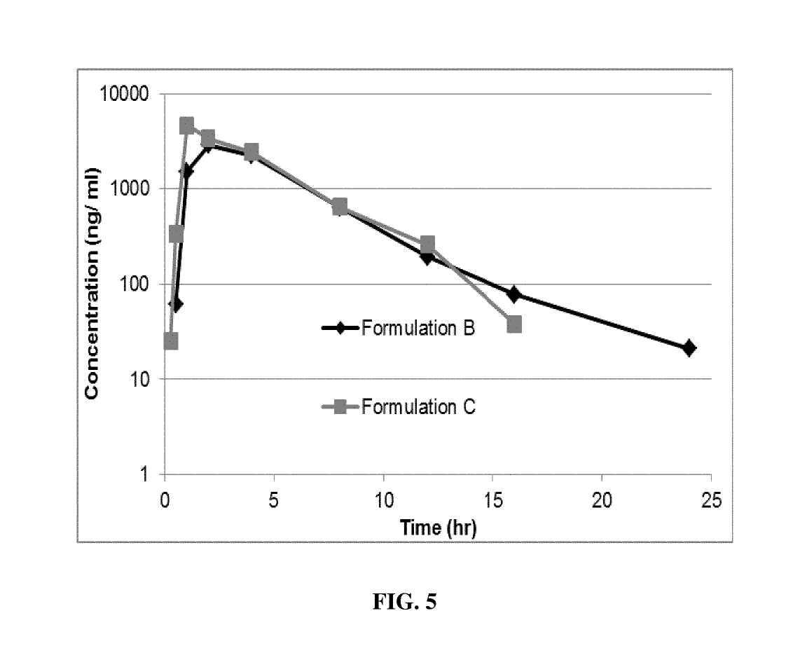

In contrast, Formulations B and C exhibit extended release PK profiles (FIG. 5 and Table 2) with 80% drug release at 4.1 hours for Formulation B and 9 hours for Formulation C.

| | AUC_{∞}/D | | Cₘₐₓ/D | | tₘₐₓ | | t_{1/2} | |
|---|---|---|---|---|---|---|---|---|
| | [ng*h*kg/ml/mg] | | [kg*ng/ml/mg] | | [hr] | | [hr] | |
| formulation | mean/median | stdev | mean/median | stdev | mean/median | stdev | mean/median | stdev |
| Formulation B | 836 | | 161 | | 2.0 | | 1.0 | |
| Formulation C | 836 | 116 | 219 | 96 | 1.0 | 0 | 1.8 | 0.5 |

We claim:

1. A pharmaceutical composition in the form of a tablet comprising: (i) dimethyl fumarate as an active substance, wherein the active substance is present in the amount of 30-90% by weight of the tablet, and (ii) one or more extended release polymer matrix present in the amount of 1-70% by weight of the tablet, wherein the active substance is distributed throughout the matrix and wherein the tablet has an average of the length and the width in the range of 3.5-6.5 mm.

2. The pharmaceutical composition of claim 1, wherein the average of the length and the width is in the range of 3.5-4.5 mm, 3.6-4.4 mm, 3.7-4.3 mm, 3.8-4.2 mm, 3.9-4.1 mm, 4.5-5.5 mm, 4.6-5.4 mm, 4.7-5.3 mm, 4.8-5.2 mm, 4.9-5.1 mm, 5.5-6.5 mm, 5.6-6.4 mm, 5.7-6.3 mm, 5.8-6.2 mm or 5.9-6.1 mm.

3. The pharmaceutical composition of claim 1, wherein the average of the length and the width is 4.0 mm or 6.0 mm.

4. The pharmaceutical composition of claim 1, wherein the tablet has a thickness of 1-3 mm.

5. The pharmaceutical composition of claim 4, wherein the tablet has a thickness of 1-2 mm.

6. The pharmaceutical composition of claim 1, wherein the extended release polymer is selected from the group consisting of hydroxylpropyl methyl cellulose (HPMC), ethyl cellulose (EC), hydroxypropyl cellulose (HPC), polyvinylpyrrolidone (PVP), polyethylene oxide (PEO), glyceryl monostearate, SoluPlus, polyvinyl alcohol (PVA), hydroxypropylmethylcellulose acetate succinate (HPMCAS), ethylene vinyl acetate (EVA), methacrylates, cellulose acetate butyrate (CAB), cellulose acetate phthalate (CAP), poly(ethylene glycol), poly(vinyl acetate) (PVAc), polylactide (PLA), polyglycolide (PGA), copolymers of PLA/PGA and polycaprolactone (PCL), polyvinylpyrrolidone-co-vinyl acetate (Kollidon VA-64), polyrethanes, poly(lactic acid), poly(glycolic acid), poly(anhydride-imides), Poly(anhydride-esters), poly(iminocarbonates), poly(phosphazenes), poly(phosphoesters), alginic acid, carbomer copolymer, carbomer homopolymer, carbomer interpolymer, carboxymethylcellulose sodium, carrageenan, cellaburate, ethylcellulose aqueous dispersion, ethylcellulose dispersion type B, glyceryl monooleate, guar gum, hydroxypropyl betadex, polyvinyl acetate dispersion, shellac, sodium alginate, starch, pregelatinized starch and pregelatinized modified xanthan gum.

7. The pharmaceutical composition of claim 6, wherein the extended release polymer is HPMC.

8. The pharmaceutical composition of claim 1, wherein the active substance is present in the amount of 60-70% by weight of the tablet.

9. The pharmaceutical composition of claim 1, wherein the extended release polymer is present in the amount of 10-20% by weight of the tablet.

10. The pharmaceutical composition of claim 1, wherein the tablet is further coated with an enteric coating.

11. The pharmaceutical composition of claim 10, wherein the enteric coating comprises an excipient selected from the group consisting of a copolymer of methacrylic acid and methyl methacrylate, a copolymer of methacrylic acid and ethyl acrylate, hypromellose phthalate (HPMCP), cellulose acetate phthalate.

12. The pharmaceutical composition of claim 11, wherein the enteric coating comprises a copolymer of methacrylic acid and methyl methacrylate.

13. The pharmaceutical composition of claim 12, wherein the ratio of methacrylic acid to methyl methacrylate in the copolymer is about 1:1.

14. The pharmaceutical composition of claim 13, wherein the enteric coating further comprises a plasticizer.

15. The pharmaceutical composition of claim 14, wherein the plasticizer is triethyl citrate.

16. The pharmaceutical composition of claim 10, wherein the enteric coating is present in the amount of 1-20% by weight of the tablet.

17. The pharmaceutical composition of claim 1, wherein the tablet further comprises a lubricant selected from the group consisting of behenoyl polyoxylglycerides, calcium stearate, hydrogenated castor oil, hydrogenated coconut oil, glyceryl behenate, glyceryl monostearate, glyceryl tristearate, lauric acid NF32, magnesium stearate, light mineral oil, myristic acid, hydrogenated palm oil, palmitic acid, poloxamer, polyethylene glycol, polyoxyl 10 oleyl ether, polyoxyl 15 hydroxystearate, polyoxyl 20 cetostearyl ether, polyoxyl 35 castor oil, hydrogenated polyoxyl 40 castor oil, polyoxyl 40 stearate, polysorbate 20, polysorbate 40, polysorbate 60, polysorbate 80, potassium benzoate, sodium benzoate, sodium lauryl sulfate, sodium stearate, sodium stearyl fumarate, sorbitan monolaurate, sorbitan monooleate, sorbitan monopalmitate, sorbitan monostearate, sorbitan sesquioleate, sorbitan trioleate, stearic acid, stearic acid, purified sucrose stearate, Talc, hydrogenated vegetable oil type I and zinc stearate.

18. The pharmaceutical composition of claim 1, wherein the tablet further comprises a glidant selected from the group consisting of calcium phosphate tribasic, calcium silicate, powdered cellulose, magnesium oxide, magnesium silicate, magnesium trisilicate, dental-type silica, hydrophobic colloidal silica, colloidal silicon dioxide, sodium stearate and Talc.

19. A pharmaceutical composition in the form of a capsule comprising one or more tablets of claim 1.

20. A method of treating a subject having multiple sclerosis comprising administering to the subject an effective amount of a pharmaceutical composition of claim 1.
